(19) United States
(12) Patent Application Publication
(10) Pub. No.: US 2014/0087326 A1
(43) Pub. Date: Mar. 27, 2014

(54) APPARATUS AND METHOD FOR DENTAL IMPLANT FIXTURE LOCATION DETERMINATION

(71) Applicants: Mark Reifman, Rishon Le Zion (IL); Abraham Taub, Rishon Le Zion (IL)

(72) Inventors: Mark Reifman, Rishon Le Zion (IL); Abraham Taub, Rishon Le Zion (IL)

(21) Appl. No.: 13/510,913

(22) PCT Filed: Oct. 20, 2010
(86) PCT No.: PCT/IL10/00864

§ 371 Date: Jun. 18, 2013

Related U.S. Application Data
(60) Provisional application No. 61/263,935, filed on Nov. 24, 2009.
(60) Provisional application No. 61/308,419, filed on Feb. 26, 2010.

(51) Int. Cl. A61C 8/00; A61B 19/00; A61B 5/05; A61C 19/04

(52) U.S. Cl. CPC A61C 8/0089; A61C 19/04; A61B 19/54; A61B 5/05
USPC 433/27; 433/173

(57) ABSTRACT

An apparatus for accurate dental implant fixture location determination including an inductive Eddy current effect based dental implant fixture location sensor and a handle. For detection and determination of the dental implant fixture location, the apparatus with the sensor is inserted into a patient mouth and moved in a scanning motion over the gums into which one or more dental implant fixtures have been earlier inserted.

### CROSS-REFERENCE TO RELATED APPLICATIONS

This application, which is a non-provisional application being filed under 37 CFR 1.53(b) and 35 USC 111, claims the benefit of the priority date of the United States Provisional Application for patent filed on Nov. 24, 2009 and assigned Ser. No. 61/263,935 and of the United States Provisional Application for patent filed on Feb. 26, 2010 and assigned Ser. No. 61/308,419 which are hereby incorporated by reference.

### TECHNOLOGY FIELD

The present apparatus and method generally relate to dental restorative or corrective work. In particular, the present apparatus relates to an apparatus for identification of implant fixture location and marking the location for further dental restorative and corrective work.

### BACKGROUND

In the field of dentistry, it is often necessary to replace native teeth by prosthetic teeth, mounted on one or more dental implants to maintain an individual ability to digest food and his or her cosmetic appearance. Dental implants are increasingly used in such procedures. Dental implant is typically composed of a metallic fixture, covered with a metallic plug or cup and it is anchored within the maxillary or mandibular bone. For implant fixture insertion the gum tissue should be opened and then holes are drilled in the patient's jaw and the implant fixtures are fixed, typically screwed, into the holes. The gum tissue is than stretched and sutured over the fixtures. At a later stage the implant fixtures receive a post that bears a thread with the help of which prosthetic teeth are attached to the post and fixed over the post.

Dental practitioners usually insert the post when the implant fixture sufficiently integrates with the recipients' jaw, a process that takes a few months. In order to determine the implant fixture location the practitioner has to make a long cut in the gum unveiling the fixtures. The plug is replaced by another plug or cover enhancing the gum healing process and a new suture enabling unobstructed access to the fixtures is made. Repeat surgical interventions increase life risk to operation sensitive patients such as patients suffering from diabetes and having poor blood coagulation, or patient suffering from different paroxysmal phenomena such as hypertonia or hypotonia, patients with pacemakers, and others.

Availability of certain procedures and apparatuses for precise location of implant fixtures will allow to open a very small area of gum tissue exactly above the surgery cup of each implant fixture and to avoid procedures of cutting and then suturing up of gum tissue, and reduce the number of surgical interventions. Furthermore, disposable apparatuses for precise location of implant fixtures will simplify the procedure for the practitioner by making sterilization of the apparatus unnecessary.

### SUMMARY

An apparatus for accurate dental implant fixture location determination including an inductive eddy current effect based dental implant fixture location sensor and a handle.

The dental implant fixture location sensor is a Planar Printed Circuit Board inductive coil the windings of which are arranged on a single plane and all of the windings participate in sensing the eddy current. The sensor may be a reusable or disposable sensor.

For detection and determination of the dental implant fixture location, the apparatus with the sensor is introduced into a patient mouth and moved in a scanning motion over the gums into which one or more dental implant fixtures have been earlier inserted. Upon determination of the dental implant fixture location a marking accessory may be operated to mark the location and assist the dental practitioner in making a smaller gum incision and reducing the subject healing time.

### GLOSSARY

The term “eddy current” as used in the present disclosure means alternating electrical currents, usually of high frequency, which can be induced to flow in any metallic materials.

The term “Q-factor” or quality factor as used in the present disclosure has its conventional meaning of the quality factor of an inductor, which is the ratio of inductors inductive reactance to its resistance at a given frequency, and is a measure of its efficiency.

The term “Planar Printed Circuit Board Inductive Coil” as used in the present disclosure relates to a single-layer or multiple-layer printed circuit board (PCB) structure having a thickness of up to 2 mm.

The term “Leading Edge” as used in the present disclosure relates to the first blunt edge of the guide, touching the flexible part or tongue of the sensor when inserting the sensor into the handle of the apparatus.

The term “Trailing Edge” as used in the present disclosure relates to the second sharp edge of the guide, cutting the flexible part of the sensor when extracting the sensor from the handle of the apparatus.

### BRIEF LIST OF FIGURES

The method and apparatus disclosed are herein presented, by way of non-limiting examples only, with reference to the accompanying drawings, wherein like numerals depict the same elements throughout the text of the specifications. The drawings are not necessarily to scale, emphasis instead being placed upon illustrating the principles of the method and the apparatus.

FIG. 1 is a schematic illustration of the present apparatus for dental implant fixture location determination;

FIGS. 2A, 2B and 2C are schematic plan view and cross-section view illustrations of an exemplary embodiment of a sensor for dental implant fixture location determination;

FIGS. 3A and 3B are cross-section simplified illustrations of an exemplary embodiment of the apparatus of FIG. 1, in accordance with the current method and apparatus;

FIGS. 4A, 4B, 4C, 4D and 4E are cross-section simplified illustrations of an exemplary method of use of the embodiment of the apparatus of FIGS. 3A and 3B;

FIG. 5 is a block diagram of the controller of the present apparatus for dental implant fixture location determination;

FIG. 6 is a schematic illustration of a method of an exemplary use of the apparatus for dental implant fixture location determination;

FIGS. 7A-7C are schematic illustrations of additional exemplary embodiments of a disposable sensor for dental implant fixture location determination; and

FIG. 8 is a simplified cross section view illustration of another method for marking the location of an implant fixture in accordance with the present apparatus for dental implant fixture location determination.

### DETAILED DESCRIPTION OF EXEMPLARY EMBODIMENTS

In the following detailed description, for purposes of explanation only, numerous specific details are set forth in order to provide a thorough understanding of the present apparatus and method. It will be apparent, however, that the present system and method may be practiced without these specific details. In other instances, well-known structures and devices are schematically shown in order to simplify the drawings.

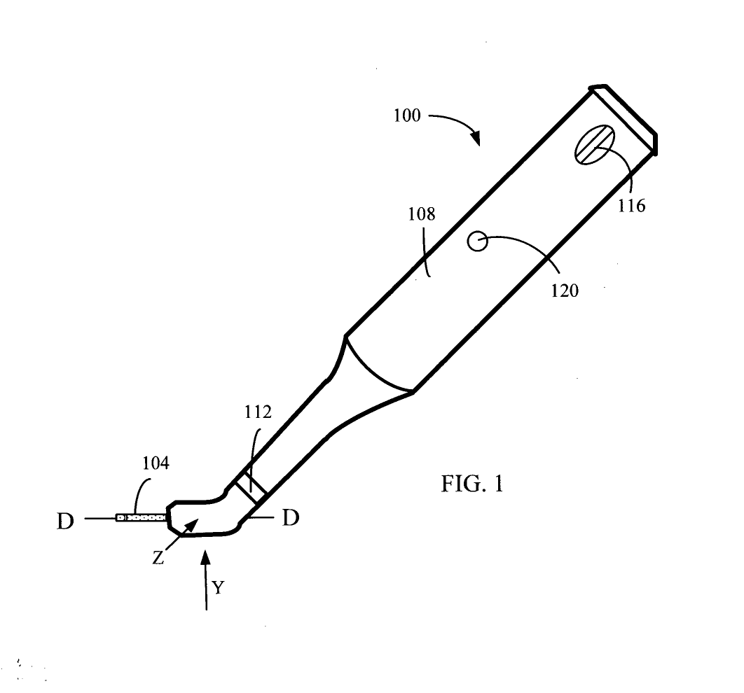

FIG. 1 is a schematic illustration of the present apparatus for dental implant fixture location determination shown in orientation operating on a mandibular bone. Apparatus 100 includes an inductive dental implant fixture location sensor 104, inserted into a handle 108, a light signal indicator 112, an audio signal indicator 116 and gum tissue marking or coagulation activation button 120. Handle 108 may be a convenient to hold, fitting a palm case containing control and processing electronics, primary or rechargeable batteries providing power to apparatus 100. Alternatively, apparatus 100 may receive power supply from a regular network receptacle. Dental fixture location sensor 104 is a detachable sensor and in use is coupled to handle 108 by inserting it into the handle. Sensor insertion provides the required electrical connection. Light signal indicator 112 may be a dual or more color Light Emitting Diode (LED) and the audio signal indicator 116 may be a simple buzzer.

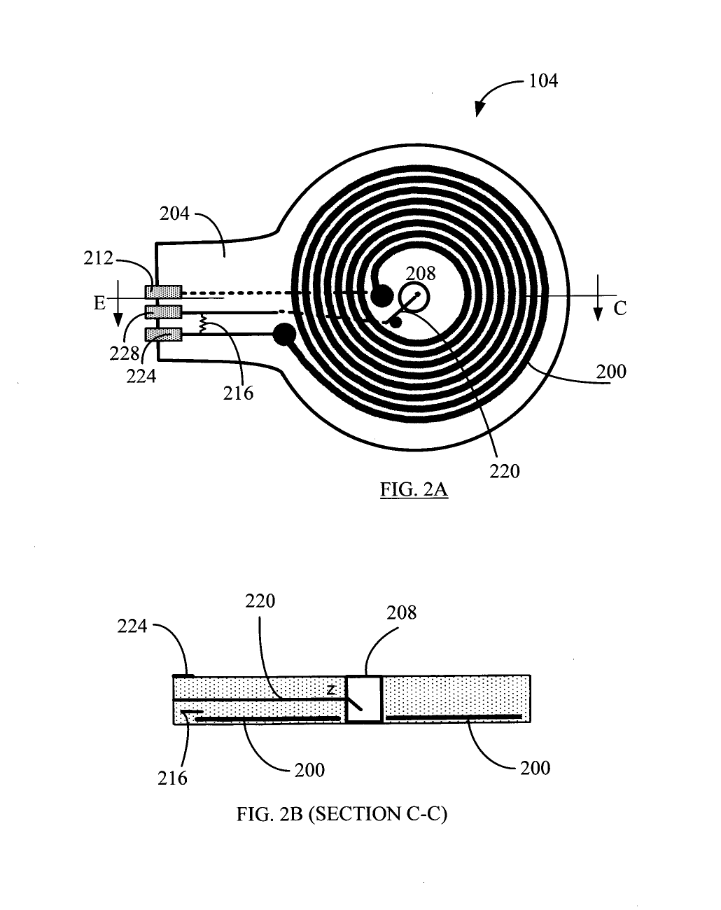

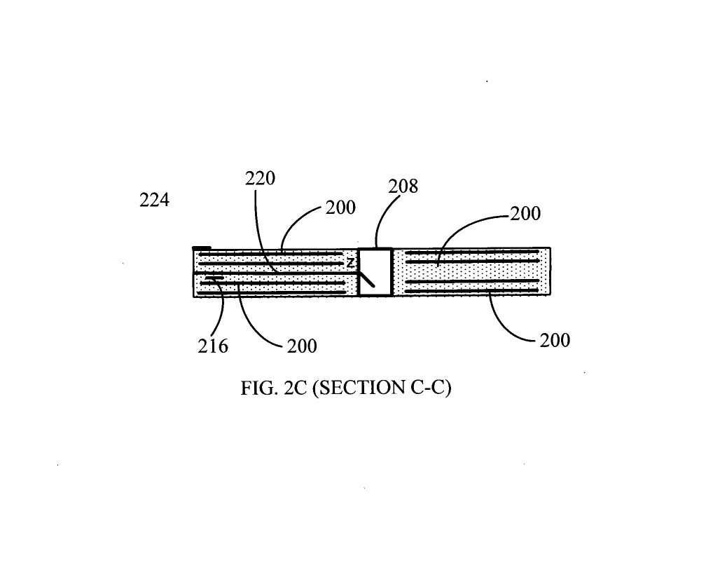

FIG. 2A is a schematic illustration of an exemplary embodiment of the present sensor for dental implant fixture location determination. Sensor 104 is a planar printed circuit board (PCB) inductive coil 200 in which all windings are located on a single plane or evenly distributed between all available PCB layers on a substrate 204 with an opening 208 in the center of the coil. Substrate 204 may be any type of bio-compatible material such as paper, plastic and other materials. Reusable sensor 104 may contain non-active, non-reactive and/or temperature-insensitive materials rendering sensor 104 fully sterilizable.

Unlike the common three-dimensional cylindrical form coil, in which only the windings close to the tissue (jaw) into which metallic implant is inserted, would participate in sensing the eddy currents, in the planar printed circuit board inductive coil where the coil windings located in different layers of the multilayer printed circuit board are very close to each other, all windings may participate in sensing eddy currents induced in dental implants. The structure described substantially increases the sensitivity of the planar printed circuit board inductive coil. For all practical purposes a multilayer planar PCB inductive coil may be considered as a sensor all windings of which are in a single plane.

A gum tissue coagulating and marking electrode 220 extends through substrate 204 to the central opening 208 of sensor 104. Electrode 220 is operative to accept through contact 228 (FIG. 2A) a voltage sufficient to coagulate the gum tissue exactly above or below (depending on the location of the fixture in the maxillary or mandibular bone) the opening leaving a visible impression or mark on the gum. The coagulating current passes between electrode 220 and patient gum/body which for all practical purposes represents a ground electrode. The arrangement of electrode 220 represents a type of marking accessory, wherein the coagulated gum spot marks exact location of the fixture.

Depending on the desired sensitivity the sensor implementation may be a single layer (FIG. 2B) or a multilayer (FIG. 2C) planar printed circuit board inductive coil in which all windings are arranged on a single plane. When sensor 104 is inserted into handle 108 (FIG. 1), contacts 212 and 224 enable communication of the sensor coil 200 with the processing electronics located in the case of handle 108. Contacts 212 and 224 may be deposited on one or both sides of the sensor and depending on the thickness of the sensor on side surfaces of the sensor. In one embodiment of the single layer sensor 104, planar printed circuit board inductive coil 200 contacts 212 and 214 may be deposited on both surfaces of substrate 204, making sensor not sensitive to the orientation of sensor insertion into handle 108.

Sensor 104 may serve as an ON/OFF switch. Insertion of sensor 104 into handle 108 switches ON apparatus 100. Extraction of sensor 104 from handle 108 may switch OFF apparatus 100, eliminating the need in any dedicated ON/OFF switch.

In one embodiment sensor 104 is a reusable sensor, made of non-active, non-reactive and/or temperature-insensitive materials and can be readily attached/detached to/from handle 108 for sensor sterilization. In another embodiment sensor 104 is a disposable sensor. In order to avoid repeat use of the disposable sensor a fuse 216 is located in the inner layer of substrate 204. When sensor 104 is operated for the first time, mounted in the handle 108 (FIG. 1) control and processing electronics senses presence of fuse 216 through contacts 224 and 212 and identifies the disposable sensor as an original not used yet sensor. In order to avoid repeat use of the same sensor, the control electronics burns fuse 216. If a sensor with a burned fuse is inserted into the handle 108, the control and processing electronics would indicate optically and/or acoustically that a used or wrong sensor is inserted, and then disable the future operation.

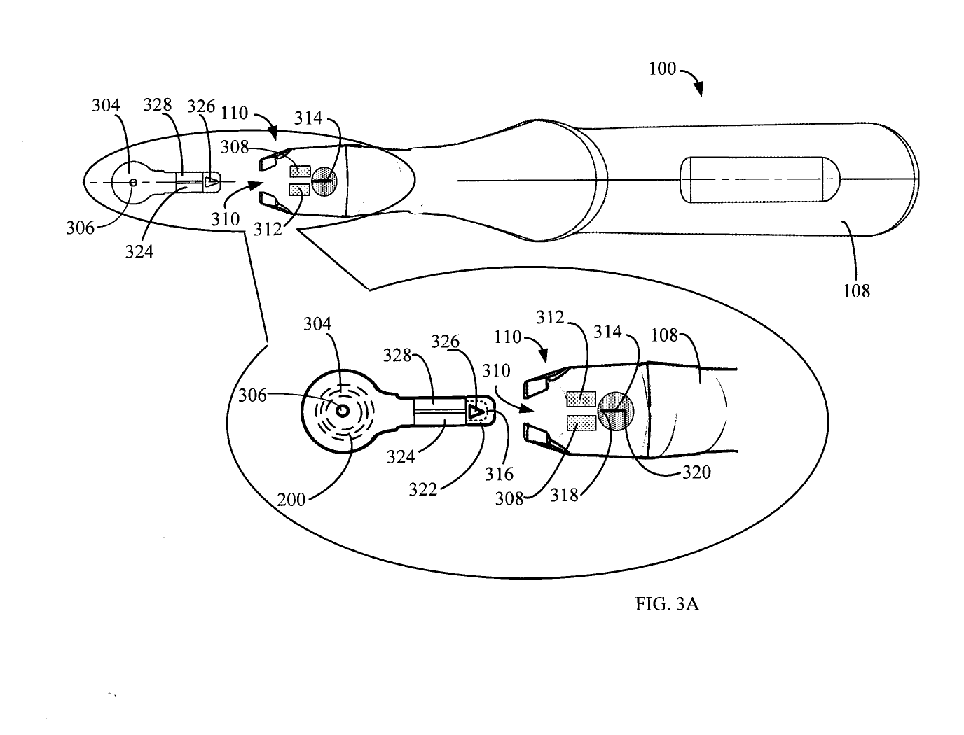

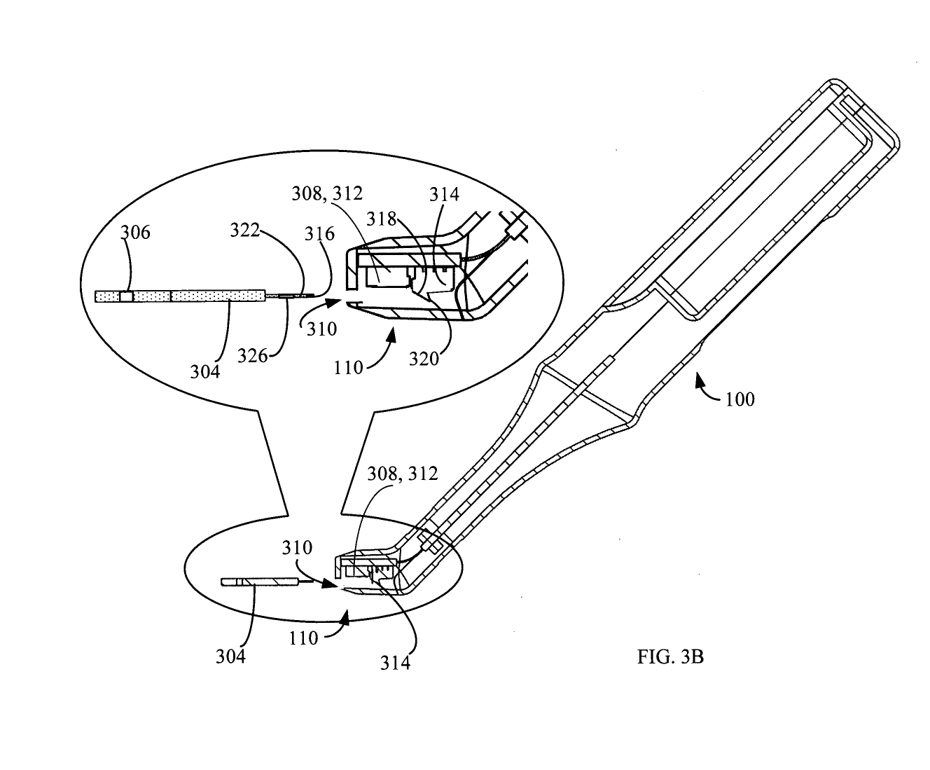

FIG. 3A is a cross section view simplified illustration of yet another embodiment of the apparatus 100 and a sensor 304. Sensor 304 may be inserted into head 110 of handle 108 of FIG. 1 and different views described below are taken along the axis D-D and viewed from the direction indicated by arrow Y of FIG. 1. Unlike FIG. 1, in FIG. 3A sensor 304 is not yet inserted into head 110 of handle 108. Head 110 of handle 108 includes a slot 310, two electric contacts 308 and 312, and a guide 314 having a blunt leading edge 318 and a sharp trailing edge 320. Disposable sensor 304 may be a multilayer printed circuit board where two most internal layers are made of a flexible material. These flexible layers extend from the rigid segment of sensor 304 and form a flexible tongue 322. The rigid part 304 includes a coil 200 (shown in broken lines), an opening 306 and contact pads 324 and 328 which terminate the coil 200. Flexible tongue 322 includes an aperture 326 and an electrically conducting jumper 316 being a segment of the coil 200. Jumper 316 (shown in broken lines) is located between the flexible layers of tongue 322. The jumper location defines the border of flexible tongue 322 and in particular the leading edge of the tongue.

FIG. 3B is a cross section view simplified illustration of apparatus 100 of FIG. 1 and sensor 304 taken along the axis D-D and viewed from the direction indicated by arrow Z, perpendicular to that indicated by arrow Y. In FIG. 3B sensor 304 is not yet inserted into head 110 of handle 108. Insertion of sensor 304 into handle 108 switches ON apparatus 100. When sensor 304 is implemented as a reusable sensor, it may not include internal flexible layers and the flexible tongue 322 with jumper 316 and aperture 326.

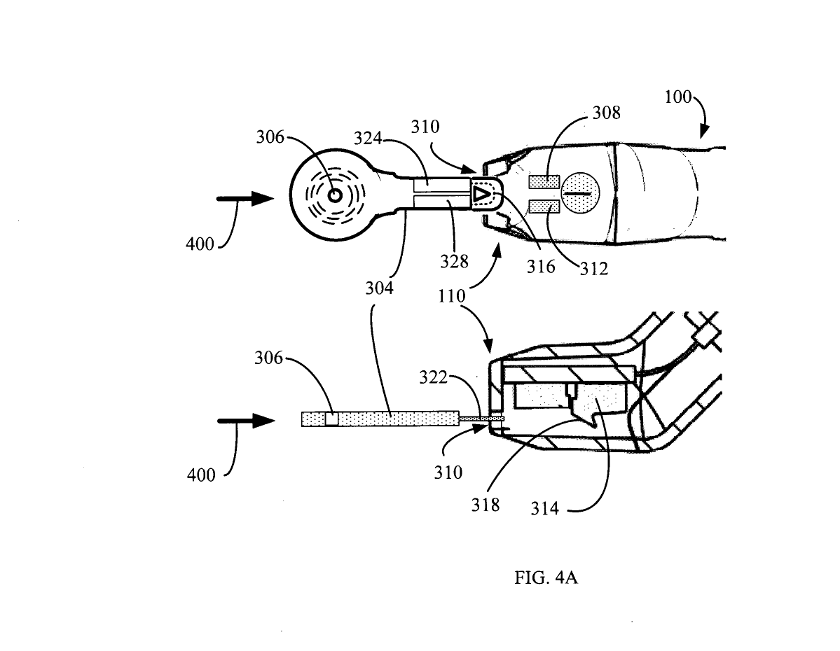

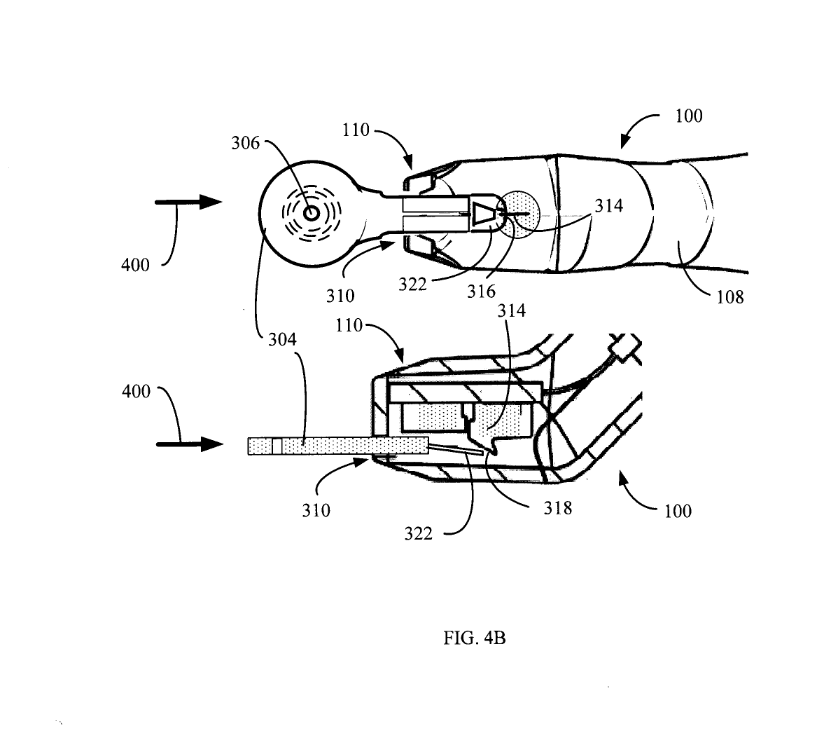

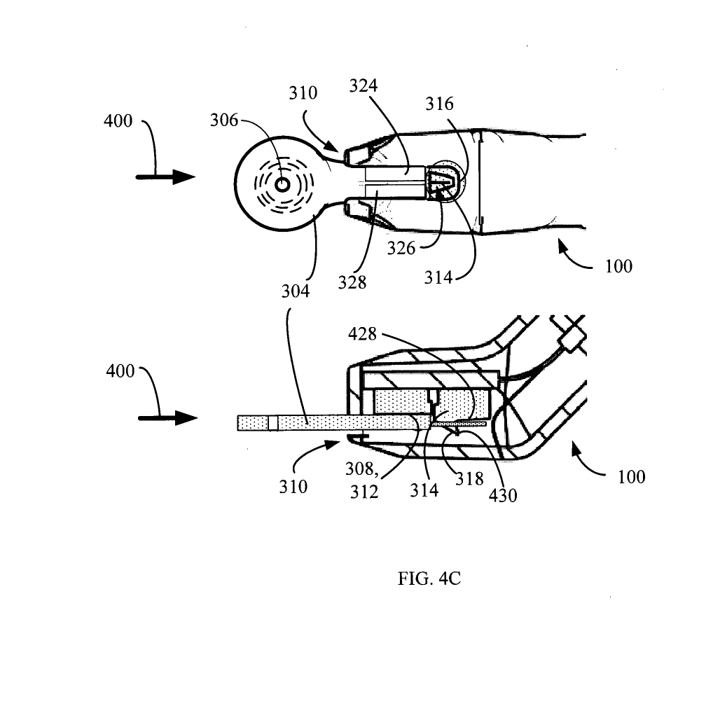

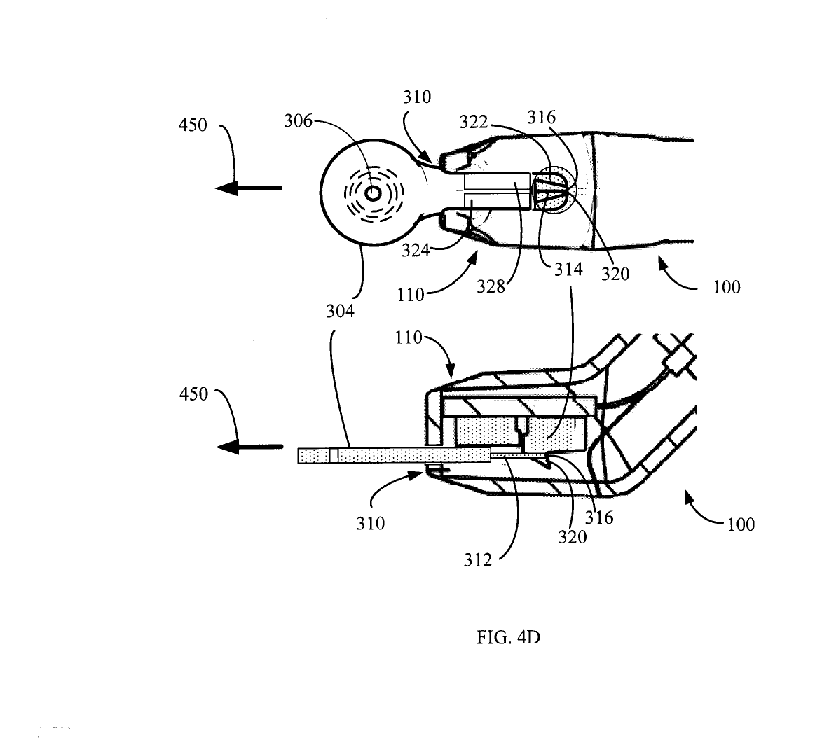

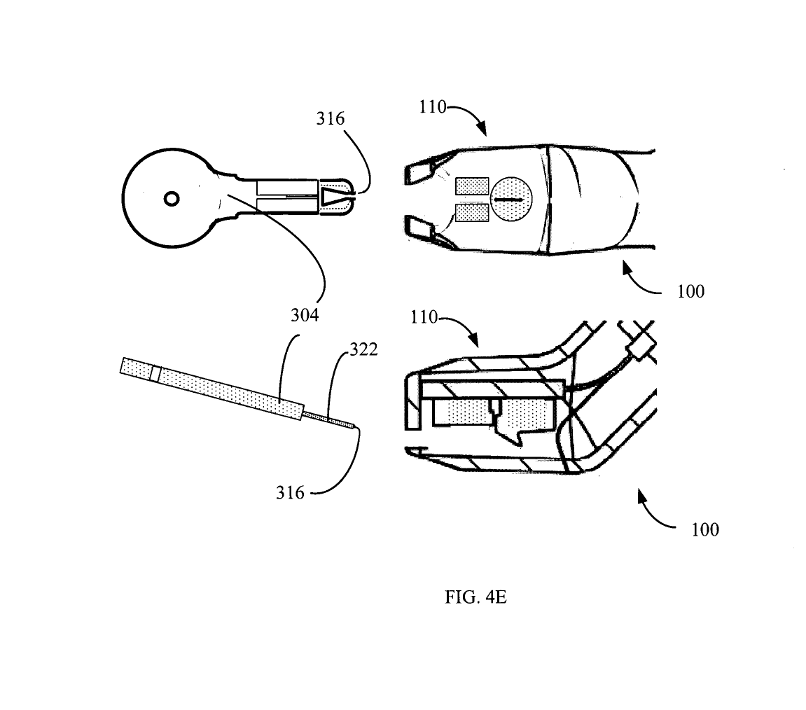

FIG. 4 illustrates the method of insertion of sensor 304 into head 110 of apparatus 100 followed by removal of sensor 304 from head 110 of apparatus 100 during which the jumper 316 is severed rendering disposable sensor 304 non-reusable. Pairs 4A, 4B, 4C and 4D, are each cross section view simplified illustrations of apparatus 100 head 110 and sensor 304 of FIGS. 3A and 3B.

Reference is now made to FIG. 4A, in which sensor 304 is partially inserted into slot 310 of head 110 in the direction indicated by arrow 400. As shown in FIG. 4B, further insertion of sensor 304 into slot 310 of head 110 brings tongue 322 in touch with blunt edge 318 of the guide 314, which urges flexible tongue 322 downward while gliding along and over blunt leading edge 318 of guide 314.

As shown in FIG. 4C, further insertion of sensor 304 into slot 310 of head 110 brings tongue 322 of sensor 304 with jumper 316 to further glide beyond apex 430 of blunt leading edge 318 of guide 314 and drop coming to rest, trapped at the base 428 of guide 314, which, at this point, protrudes through aperture 326. Contacts 324 and 328 of sensor 304 establish electrical communication with respective contacts 308 and 312 of head 110.

Following implant fixture location determination and marking, sensor 304 is removed from head 110 slot 310 by pulling sensor 304 in the direction indicated by arrow 450 (FIG. 4D). Removing sensor 304 cuts jumper 316, trapped at the base of guide 314 against sharp trailing edge 320, permanently damaging coil 200 and rendering sensor 304 unusable.

FIG. 4E shows severed jumper 316 of sensor 304 which is fully removed from handle 108 head 110. At this point sensor 304 may be discarded.

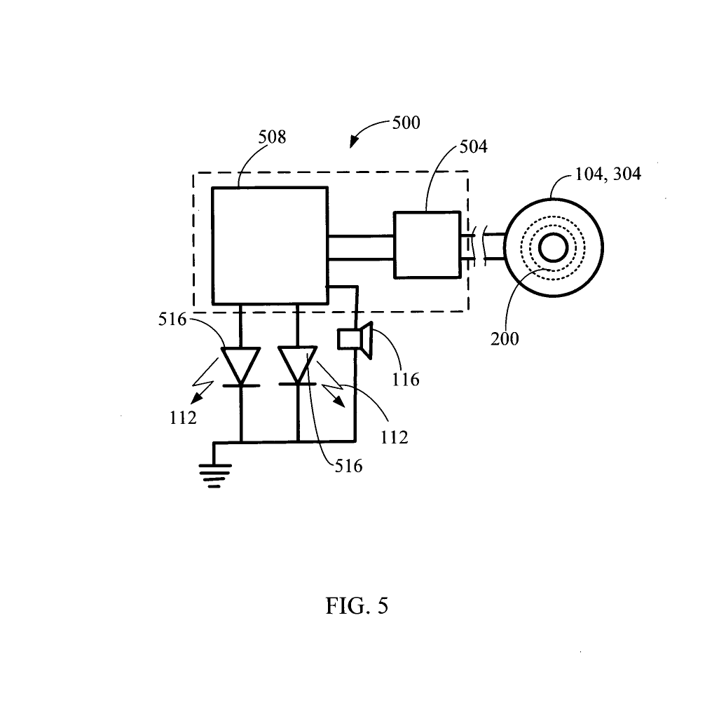

FIG. 5 is a block diagram of the controller of the present apparatus for dental implant fixture location determination. Controller 500 includes a self-oscillating generator 504 inductive element of which is coil 200 of sensor 104 or 304. Generator 504 produces a certain frequency measured by microprocessor 508. Microprocessor 508 is receiving and processing the frequency generated by generator 504, analyzes the signal and controls operation of the light indicator 112 and/or audio indicator 116 (FIG. 1). Light indicator 112 may be a dual or more color LED 516.

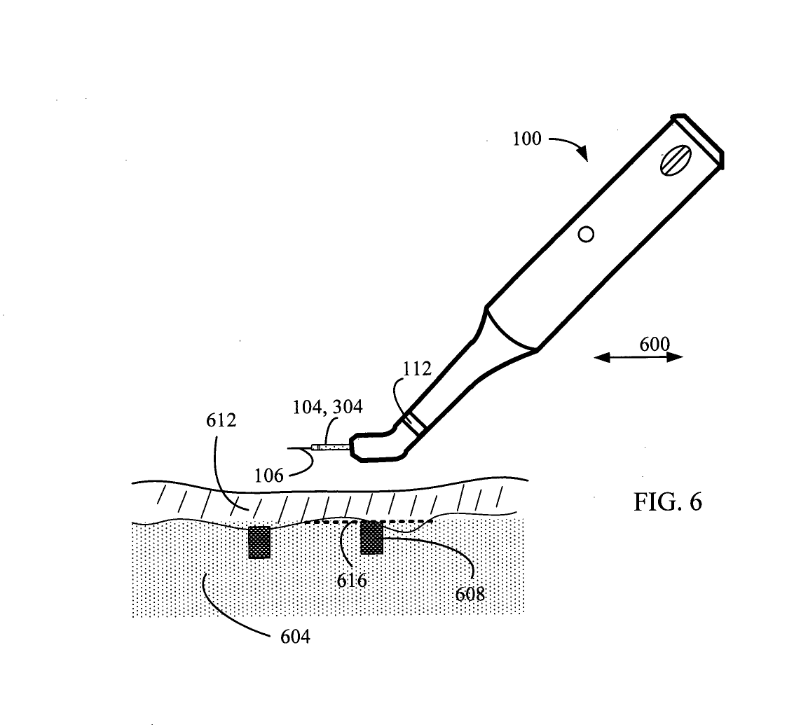

For detection and determination of the dental implant fixture location, as shown in FIG. 6, apparatus 100 with the sensor 104 or 304 is introduced into a patient mouth and moved in a scanning motion as schematically shown by arrow 600 relative to jaw 604 into which one or more dental implant fixtures 608 have been earlier inserted. Sensor 104 or 304 is positioned so that the surface 106 of two-dimensional coil 200 is about to be as parallel as possible to the expected plane defined by the upper surface 616 of fixture 608. As explained above, a layer of gum tissue 612 covers the fixtures location and the fixtures cannot be visually detected. The sensor coil 200 is a part of a self-oscillating generator 504, which converts the Q-factor of the coil 200 affected by the presence of fixture 608 according to the eddy currents, induced in upper surface 616 of fixture 608 into appropriate frequency. The frequency is a function of distance between the centers of the sensor coil 200 and the nearest implant fixture 608 surface 616.

Indicators, such as audio signal indicators may be employed to sound an audio signal indicating the proximity of sensor 104 (FIG. 1) or 304 (FIG. 3) to the target fixture. For example, control and processing electronics may activate audio signal indicator 116 (FIG. 1) assisting the practitioner and indicating to him how the sensor 104 or 304 is positioned relatively the dental implant fixture 608 location. This may be performed by changing the tone of the audio signal. However, light and particularly color indications have been shown to be more effective and readily accepted by users.

Light indicator 112 and in particular the color of the light emitted by the light indicator may be used to indicate the relative proximity of the coil 200 with respect to the fixture 608. For example, a blinking light may be indicative of movement of sensor 104 or 304 relative to dental implant fixture 608, whereas a continuous (non-blinking) light may be indicative of sensor 104 or 304 being stationary relative to dental implant fixture 608.

Additionally, a green blinking light indicator 112 may be operative to indicate that sensor 104 or 304 is approaching the center of implant fixture 608, while a red blinking light indicator 112 may be operative to indicate that sensor 104 or 304 is distancing from fixture 608. A blue light, for example, may become operative when sensor 104 or 304 is centered directly over the target. Alternatively, indicator 112 may stop blinking and become turned on continuously emitting green light when opening 208 (FIG. 2) is located at the closest to the center of insert 608 location.

When based on the light or audio signal the practitioner identifies the dental implant fixture 608 location he or she pushes button 120 (FIG. 1) and activates supply of voltage to gum tissue coagulation electrode 220 (FIG. 2) marking the exact identified dental implant fixture location, by coagulating a small area (or similar) of gum tissue to create a small mark.

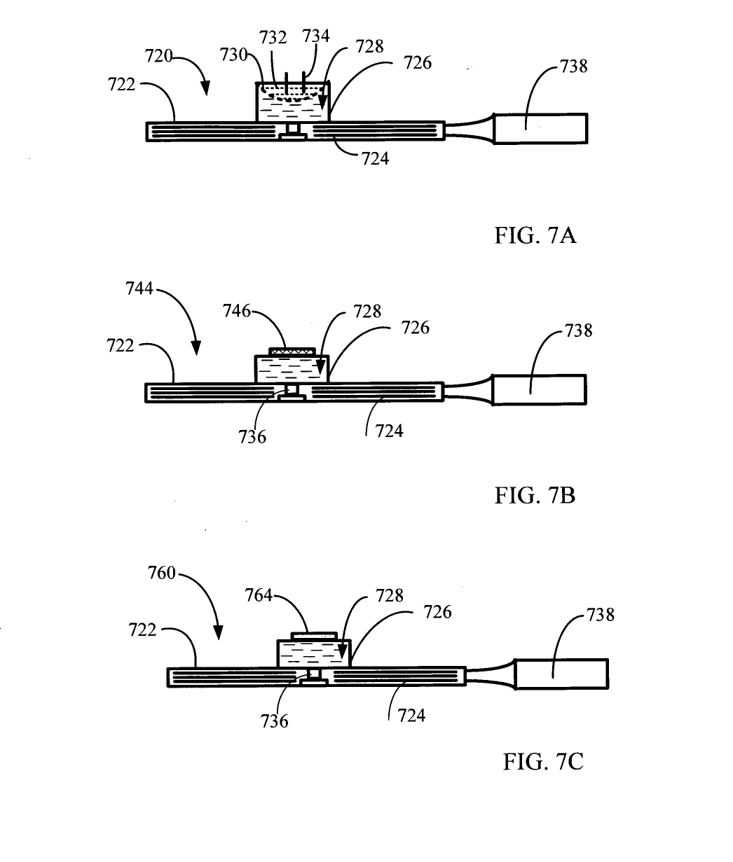

In an alternative embodiment, the practitioner may introduce through opening 208 a drop of a marking substance, which may be ink or similar material, marking the exact identified dental implant fixture location. FIGS. 7A-7C are schematic illustrations of additional exemplary embodiments of a disposable sensor for dental implant fixture location determination. In one embodiment, shown in FIG. 7A, disposable sensor 720 includes a flat body 722 made of suitable material with a planar printed circuit board inductive coil 724, and a miniature tank 726 containing a marking fluid 728 such as ink. Tank 726 may have a section or compartment separated from the ink section by a flexible fluid impermeable film 730 filled in by another fluid 732 for example, water. A pair of electrodes 734 is inserted into the compartment. The electrodes are in contact with the water. One of the tank 726 walls includes a small orifice 736 with a capillary channel having a diameter of 20 micron to 200 micron. Connector 738 inserts into head 110 of handle 108 (FIG. 1) and provides an electrical communication path with the control and processing electronics and a source of power located in the handle or in a separate power unit.

Upon detection of a fixture location, locator 100 communicates the location to the dental practitioner by light 112 and/or audio signal 116 indicators, the practitioner pushes button 120 (FIG. 1) connecting a source of current to electrodes 738. A process of hydrolyses in the water takes place and released gases bend flexible diaphragm 730 outwardly increasing pressure in tank 726. The pressure releases marking substance 728 making an impression on the gum 612 and indicating to the practitioner exact implant fixture 608 (FIG. 6) location.

The size of spot formed by the released marking substrate 728 is about 1 mm to 3 mm and depends on the preferences of the practitioner. The thickness of the ink layer should be sufficient to form a spot with density clearly distinguishable on the background of the surrounding gum.

In another embodiment, shown in FIG. 7B, a disposable sensor 744 includes a miniature tank 726 containing a marking fluid 728 and a current operated heat source 746. The source may be immersed in marking fluid 728 or as shown in FIG. 7B external, attached to one of the walls of tank 726. Upon detection of fixture location communicated to the dental practitioner by light 112 and/or audio signal 116 indicators, the practitioner pushes button 120 (FIG. 1) connecting a source of current to heat source 746. Heat generated by the source causes rapid expansion of marking fluid volume in tank 726. Volume expansion builds-up pressure that ejects or releases through nozzle 736 a dose or droplet of marking substance 728 making an impression on the gum 612 and indicating to the practitioner exact implant fixture 608 (FIG. 6) location and forming a marking spot with a diameter of about 1 mm to 3 mm.

In a further embodiment, shown in FIG. 7C, disposable sensor 760 includes a piezoelectric actuator 764. Operation of the actuator may cause ejection of one or more drops of marking fluid 728 towards the gum. In order to avoid repeat use of any one of the disposable sensors of FIGS. 7A through 7C a fuse similar to fuse 316 (FIG. 3) may be incorporated in each of the disposable sensor embodiments, although the ink reservoir may be made to contain a limited amount of ink, for example, amount sufficient for marking three or five fixture locations.

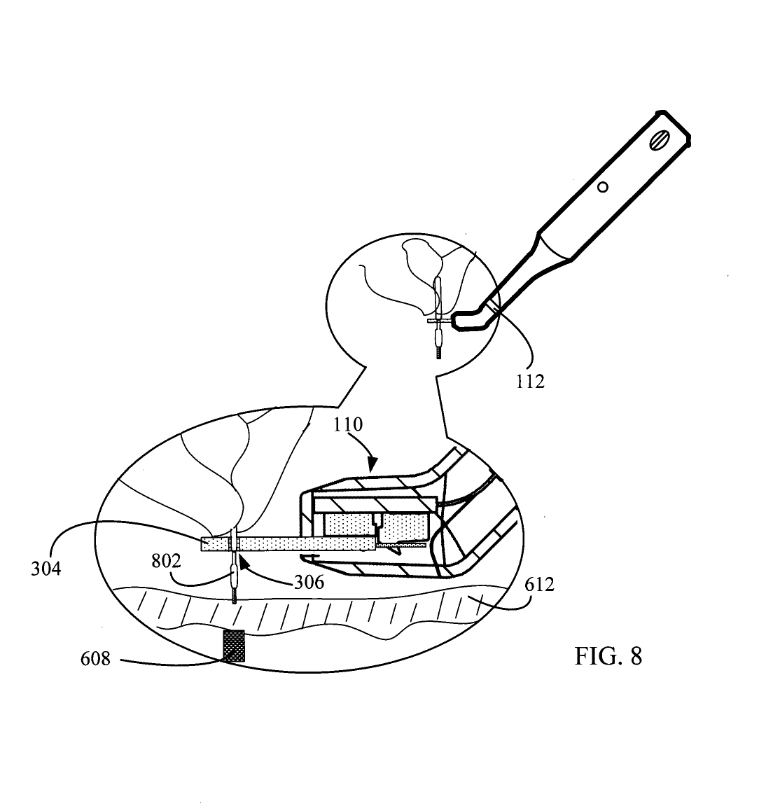

Referring now to FIG. 8, which is a simplified cross section view illustration of another method for marking the location of an implant fixture in accordance with the present apparatus for dental implant fixture location determination. Upon location of the implant fixture 608 and in order to mark the detected location, the user may hold sensor 304 in position as determined, for example, by the color of the light emitted by light indicator 112. A marking blade 802, such as a thin 1 mm diameter disposable tube-like blade, similar to a known in the art biopsy punch, may be inserted through opening 306 in sensor 304 and driven through the gum 612 over implant fixture 608 to mark the exact location of fixture 608.

The apparatus described enables fast, reliable, and simple identification of the dental implant fixture location. The practitioner has to perform a small incision in order to access the plug 616 and replace it with a similar healing cup or insert the prosthesis holding abutment.

Accurate determination of fixture location minimizes the number of surgical interventions, reduces risk to operation sensitive patients such as patients suffering from diabetes and having poor blood coagulation, or patient suffering from different paroxysmal phenomena such as hypertonia or hypotonia patients with pacemakers.

A number of embodiments have been described. Nevertheless, it will be understood that various modifications may be made without departing from the spirit and scope of the method. Accordingly, other embodiments are within the scope of the following claims:

What is claimed is:

1. An apparatus for accurate dental implant fixture location determination, said apparatus comprising:
an inductive eddy current effect based dental implant fixture location sensor;
a handle; and
wherein the dental implant fixture location sensor is a single-layer or multiple-layer planar printed circuit board inductive coil the windings of which are arranged on a single plane.

2. The apparatus according to claim 1, wherein all of the windings participate in sensing said eddy current.

3. The apparatus according to claim 1, wherein the dental implant fixture location sensor is at least one of a group consisting of reusable or disposable sensors.

4. The apparatus according to claim 3, wherein the reusable dental implant fixture location sensor is readily sterilizable.

5. The apparatus according to claim 1 wherein the dental implant fixture location sensor further comprises an implant fixture location marking accessory.

6. The apparatus according to claim 5 wherein the dental implant fixture location marking accessory is at least one of a group consisting of a marking substance, gum coagulating voltage or a thin tube.

7. The apparatus according to claim 6 wherein the marking substance is ink contained in the marking accessory in an amount sufficient to make at least one impression.

8. The apparatus according to claim 1 wherein disposable dental implant fixture location sensor further includes a fuse preventing repeat use of the disposable sensor.

9. The apparatus according to claim 1 wherein said handle also comprises a varying color LED light indicator operative to indicate movement and the relative proximity of said dental implant fixture location sensor to the fixture.

10. The apparatus according to claim 1 further comprising control and processing electronics located in the handle.

11. The apparatus according to claim 1 further comprising a dental implant fixture location sensor serving as the apparatus ON/OFF switch.

12. A method for accurate dental implant fixture location determination, said method comprising:
scanning a gum of a patient by a dental implant fixture location sensor; and
identifying based on a visual or audio signal the location detected as a dental implant fixture location.

13. The method according to claim 12, wherein said visual signal is produced by a varying color LED indicator.

14. The method according to claim 12 wherein the sensor is an inductive sensor.

15. The method according to claim 12 further comprising marking the identified dental implant fixture location.

16. The method according to claim 15 wherein the marking means are forming a mark by gum tissue coagulation.

17. The method according to claim 15 wherein marking means are forming a mark by a marking substance.

18. The method according to claim 15 wherein marking means are forming a mark by a marking blade driven through a gum.

19. An apparatus for determining and marking a dental implant fixture location, said apparatus comprising;
a disposable inductive eddy current effect based dental implant fixture location sensor for determining a dental implant fixture location; and
an implant fixture marking accessory.

20. The apparatus according to claim 19 wherein the dental implant fixture location marking accessory is at least one of a group consisting of a marking substance, gum coagulating voltage or a marking blade driven through a gum.

21. The apparatus according to claim 19 wherein the disposable dental implant fixture location sensor further includes a fuse preventing repeat use of the disposable sensor.

22. A disposable sensor for determining and marking a dental implant fixture location, said sensor comprising:
an inductive eddy current effect based dental implant fixture location sensor; and
an implant fixture marking substance tank.

23. The disposable sensor according to claim 22 further comprising a connector operative to connect the sensor to a control and processing electronics.

24. The disposable sensor according to claim 22 further comprising an actuator operative to eject the marking substance from the marking substance tank.

25. The disposable sensor according to claim 22 wherein the marking substance is ink contained in the marking substance tank in an amount sufficient to make at least one impression.

26. The disposable sensor according to claim 22 wherein disposable dental implant fixture location sensor further includes a fuse preventing repeat use of the disposable sensor.

27. A method for accurate dental implant fixture location determination, said method comprising:
providing an inductive eddy-current-effect-based dental implant fixture location sensor having a flat surface;
positioning said flat surface in parallel to the expected plane defined by the upper surface of said fixture;
scanning a gum of a patient by said fixture location sensor; and
identifying the location detected as a dental implant fixture location based on a visual or audio signal.

28. The method according to claim 27, wherein said visual signal is a varying color LED illumination.

29. The apparatus according to claim 27, wherein said disposable dental implant fixture location sensor further includes a jumper preventing repeat use of said disposable sensor, which is readily severed upon removal of said sensor from said handle by a blade located in said handle.

30. A method for preventing repeat use of a disposable dental implant fixture location sensor comprising:
providing a handle including a guide having a blunt edge and a sharp edge;
providing an inductive eddy-current-effect-based dental implant fixture location sensor having a jumper;
inserting said sensor into said handle so that said jumper glides over said blunt edge;
severing said fuse while removing said sensor from said handle by urging said fuse against said guide sharp edge.

31. A method for accurate dental implant fixture location determination, said method comprising:
scanning a gum of a patient by a dental implant fixture location sensor having an opening;
identifying based on a visual or audio signal the location detected as a dental implant fixture location; and
inserting a marking blade through said opening and into the gum so that to mark the location of said detected dental implant fixture.
